SEQUENCE LISTING (8 sequences)

SEQ ID NO: 1 - protein, 365 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 2 - protein, 797 aa, Artificial Sequence
  Description of Artificial Sequence Synthetic polypeptide
AAAMELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 3 - protein, 359 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 4 - DNA, 2401 bp, Artificial Sequence
  Description of Artificial Sequence Synthetic polynucleotide
  CDS (1)..(2391)
gcggccgccatggaattgcgtccttggcttttgtgggttgttgcagctacaggtactcttgttctgctggctgctgacgcccagggacagaaggtctttactaacacatgggcagtccggattcccggtggacccgctgtagcaaactccgtggctaggaaacacgggtttctcaacctgggccagattttcggtgactactaccacttttggcatagaggggtgaccaagcggtccctctctccccatagacccagacacagccgactgcagcgggaaccccaggtccagtggttggaacagcaggtggccaagaggagaaccaagcgggacgtgtatcaggagcctaccgatcccaaatttccacagcagtggtatctctctggggtgactcaacgtgatctgaacgtgaaggccgcttgggctcagggctacaccggtcatggaatcgtggtgagcatattggacgacggaatagaaaagaatcaccccgacttggctggaaactacgatccaggcgcatcctttgatgtcaatgatcaagatccagacccacagcctcggtacacccagatgaacgacaaccgtcatggaaccaggtgtgccggcgaagtggcagccgtcgccaataacggtgtttgcggagtgggcgttgcctacaacgcccgaatcggaggtgtgaggatgctggatggtgaagtgaccgatgccgtcgaagctcggtccttgggactgaaccccaatcatatacacatatattcagccagttggggtcctgaggatgacggcaagacagtggatggacctgcacgactcgccgaggaggctttcttccgcggcgtctctcaaggtcgcggagggctgggcagcatatttgtctgggccagtggcaacggtggccgtgaacatgactcatgtaactgtgatggctatacaaatagcatctataccctgagcatcagttccgcaactcaatttggtaacgtgccctggtactcagaggcctgctcaagcaccctcgctactacctattcatctggaaatcagaacgagaagcagatcgtcacaaccgacctgagacagaagtgtaccgaatctcatacaggcacctctgcctctgcccctctggctgccggcatcatcgctctgactcttgaagctaacaagaatcttacatggcgggatatgcaacacctggtagtacagactagtaaaccagcccatcttaacgcaaacgactgggcaacaaacggggtcggacgtaaagtatctcattcttacggatacggactgctggatgcaggagccatggtggccctcgcccaaaactggaccactgtcgctccccaaaggaagtgcataattgatatcctcactgaaccaaaagacataggcaagcggttggaggtcagaaagaccgtgaccgcctgcctgggggagcccaatcacatcacacgactcgagcacgcacaagcccgactgactctgagttataatcgacggggcgatctggctatccatctcgtcagccccatgggaaccagatccacattgttggctgctaggccccacgactacagtgctgatgggtttaacgattgggcttttatgactactcactcctgggatgaggacccaagcggagagtgggtgctggagattgaaaatacttcagaagccaataattacggcactctgaccaaatttaccctggtgttgtacgggacagcacccgagggtctgccagtgcctccagagtcatccggttgcaagactctcaccagctcccaggcctgcgtagtgtgcgaggaaggcttctccttgcatcagaagtcttgtgtccagcattgcccacccgggtttgcacctcaagtgcttgatacccactacagtacagagaatgatgttgaaacaattcgggcatccgtgtgtgccccttgtcatgcatcctgcgccacttgccagggcccagcattgaccgattgtctcagctgcccatcacacgccagcctggacccagttgaacagacctgtagccgccagtcccaatcctcacgagaaagcccaccccagcagcagccaccaagactgcccccagaagtggaggccggccagcgcctcagggcaggcttgctgccttcacacctgccagaagtagtggctggtttgagctgtgctttcatcgttcttgtatttgtcactgtttttctggttctgcagctgcggagcggattttctttccggggcgtgaaggtgtacactatggaccgtgggctcatctcctacaaaggtctgccacccgaagcctggcaggaagagtgccccagcgattccgaagaggatgaagggcgtggggaaaggactgccttcattaaggatcaatctgctctctgataagctt

SEQ ID NO: 5 - protein, 6 aa, Artificial Sequence
  Description of Artificial Sequence Synthetic peptide
RKRRKR

SEQ ID NO: 6 - protein, 4 aa, Artificial Sequence
  Description of Artificial Sequence Synthetic peptide
  MOD_RES (2)..(2): Any amino acid
  MOD_RES (3)..(3): Arg or Lys
RXXR

SEQ ID NO: 7 - protein, 105 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLER

SEQ ID NO: 8 - protein, 254 aa, Artificial Sequence
  Description of Artificial Sequence Synthetic polypeptide
IVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

(19) United States
(12) Patent Application Publication
(10) Pub. No.: US 2022/0098565 A1
(43) Pub. Date: Mar. 31, 2022

(54) PROCESS FOR MAKING RECOMBINANT ANTIDOTE TO FACTOR XA INHIBITOR

(71) Applicant: Alexion Pharmaceuticals, Inc., Boston, MA (US)

(72) Inventors: Genmin Lu, Burlingame, CA (US); Pamela B. Conley, Palo Alto, CA (US); Uma Sinha, San Francisco, CA (US)

(21) Appl. No.: 17/387,806

(22) Filed: Jul. 28, 2021

Related U.S. Application Data
(63) Continuation of application No. 16/127,864, filed on Sep. 11, 2018, now abandoned.
Application No. 16/127,864 is a continuation of application No. 15/391,020, filed on Dec. 27, 2016, now abandoned.
Application No. 15/391,020 is a continuation of application No. 14/745,901, filed on Jun. 22, 2015, now abandoned.
Application No. 14/745,901 is a continuation of application No. 13/766,652, filed on Feb. 13, 2013, now abandoned.
(60) Provisional application No. 61/598,694, filed on Feb. 14, 2012.

(51) Int. Cl. C12N 9/64

(52) U.S. Cl. CPC C12N 9/6432; C12N 9/6454; C12N 2510/02; C12Y 304/21006; C12N 9/64

(57) ABSTRACT

Disclosed are methods and isolated cells useful for the improved production of function fXa derivative protein that acts as a fXa inhibitor antidote. One aspect relates to an isolated cell comprising the r-Antidote polynucleotide and Furin polynucleotide. Another aspect relates to a method for preparing the cleaved two-chain r-Antidote by expressing, in a cell, the pre-processed r-Antidote polypeptide and a Furin polypeptide.

### CROSS REFERENCE TO RELATED APPLICATIONS

This application is a continuation of U.S. application Ser. No. 16/127,864, filed Sep. 11, 2018, which is a continuation of U.S. application Ser. No. 15/391,020, filed Dec. 27, 2016, which is a continuation of U.S. application Ser. No. 14/745,901, filed Jun. 22, 2015, which is a continuation of U.S. application Ser. No. 13/766,652, filed on Feb. 13, 2013, which claims the benefit under 35 U.S.C. § 119(e) of the U.S. Provisional Application No. 61/598,694 filed on Feb. 14, 2012, which are hereby incorporated by reference in their entirety.

### FIELD

This disclosure relates to useful cells and methods for the expression and purification of fXa derivatives.

### BACKGROUND

Anticoagulants serve a need in the marketplace in treatment or prevention of undesired thrombosis in patients with a tendency to form blood clots, such as, for example, those patients having clotting disorders, confined to periods of immobility or undergoing medical surgeries. One of the major limitations of anticoagulant therapy, however, is the bleeding risk associated with the treatments, and limitations on the ability to rapidly reverse the anticoagulant activity in case of overdosing or if an urgent surgical procedure is required. Thus, specific and effective antidotes to all forms of anticoagulant therapy are highly desirable. For safety considerations, it is also advantageous to have an anticoagulant-antidote pair in the development of new anticoagulant drugs.

Previously reported modified derivatives of fXa proteins are useful as antidotes to anticoagulants targeting fXa. The modified derivatives of fXa proteins do not compete with fXa in assembling into the prothrombinase complex, but instead bind and/or substantially neutralize the anticoagulants, such as fXa inhibitors. These modified protein derivatives, described in US Publications 2009/0098119 and 2010/0255000, require post-translational modifications for proper structure and function. Such post-translational modifications include the removal of the prepro-peptide and the cleavage of the internal -RKRRKR- (SEQ ID NO: 5) linker sequence in the fX derivative precursor to form the mature fX derivative protein.

Incomplete or inefficient processing in a host cell system can result in decreased isolation of the functional protein. Therefore, there is a need in the art for systems that improve the efficiency of processing of functional fXa derivative proteins useful as fXa inhibitor antidotes.

### SUMMARY

Disclosed herein are methods and cells for the increased production of functional r-Antidote proteins. It was previously unknown that the in vivo treatment of a human Factor Xa derivative (i.e., precursor r-Antidote) with Furin allowed for the improved processing of a functional, 2-chain protein. Accordingly, methods and cells described herein provide for improved production of functional r-Antidote by co-expression of r-Antidote and Furin in vivo.

Aspects of the disclosure relate to an isolated cell comprising:

a first polynucleotide encoding a polypeptide comprising the amino acid sequence of SEQ ID NO: 1 or a polypeptide having at least about 80% sequence identity to SEQ ID NO: 1 and

a second polynucleotide encoding a polypeptide comprising the amino acid sequence of SEQ ID NO: 2 or a polypeptide having at least about 80% sequence identity to SEQ ID NO: 2. In some embodiments, the first and second polynucleotides are on separate polynucleotide constructs, and in some embodiments, the first and second polynucleotides are on the same polynucleotide constructs, which can have separate regulatory elements.

In a related aspect, provided is a polynucleotide construct that comprises a first polynucleotide encoding a polypeptide comprising the amino acid sequence of SEQ ID NO: 1 or a polypeptide having at least about 80% sequence identity to SEQ ID NO: 1 and a second polynucleotide encoding a polypeptide comprising the amino acid sequence of SEQ ID NO: 2 or a polypeptide having at least about 80% sequence identity to SEQ ID NO: 2.

Another aspect relates to a method of preparing a cleaved two chain polypeptide comprising the amino acid sequence of SEQ ID NO: 3 or a polypeptide having at least about 80% sequence identity to SEQ ID NO: 3, wherein the method comprises, expressing in an isolated cell:

a first polynucleotide encoding a polypeptide comprising the amino acid sequence of SEQ ID NO: 1 or a polypeptide having at least about 80% sequence identity to SEQ ID NO: 1 and

a second polynucleotide encoding a polypeptide comprising the amino acid sequence of SEQ ID NO: 2 or a polypeptide having at least about 80% sequence identity to SEQ ID NO: 2.

The isolated cell may be any suitable host cell that provides for processing and cleavage of the required post-translational modifications. Suitable cells include, by way of non-limiting example, fungal cells, such as yeast cells, bacterial cells, and mammalian cells. In one embodiment, the cell is a mammalian cell or a yeast cell. In a related embodiment, the mammalian cell is a cell-type selected from the group consisting of CHO, COS, BHK, and HEK 293. In a further embodiment, the cell-type is CHO. In yet a further embodiment, the CHO cell-type is of the subtype K, M, or DG44.

### BRIEF DESCRIPTION OF THE FIGURES

FIG. 1 depicts the optimized human Furin cDNA sequence and translated amino acids. The translated amino acid sequence is referred to herein as SEQ ID NO: 2. The cDNA sequence depicted represents SEQ ID NO: 4.

FIG. 2A-B shows the effect of the Furin transfection described in Example 2 on 14G1 (expression level of the antidote: FIG. 2A and functional activity: FIG. 2B). The 3%, 10%, 30%, and 100% refer to the percentage of Furin containing plasmid relative to the total DNA transfected. GFP in FIG. 2 refers to Green Fluorescent Protein. The expression level was measured by an Enzyme-linked immunosorbent assay (ELISA) using an antibody that recognizes both the single-chain and the double-chain antidote molecules. The functional activity was measured by a fXa chromogenic activity assay in the presence of a fXa inhibitor betrixaban. Only properly cleaved r-Antidote molecule is able to bind betrixaban and neutralize its inhibitory activity toward fXa.

FIG. 3 demonstrates the effect of the Furin transfection described in Example 2 on 14G1 expression and processing of antidote (quality by Western Blotting using LC (light chain) antibody). The protein quality as assessed by Western Blots indicates that transfection of Furin completely eliminated the single-chain (SC) r-Antidote precursor.

FIG. 4 shows the transient transfection of Furin with alternative fX derivative constructs (Quality by Western Blotting). As described in Example 2, co-transfection with Furin did not improve the cleavage efficiency of the des-Gla fX derivative.

FIG. 5A-B shows the expression level (FIG. 5A) and functional activity (FIG. 5B) of antidote protein in 14G1-Furin mini-matrix experiment (clone #92, #94) described in Example 3.

FIG. 6A-D depicts the expression level (FIG. 6A) and functional activity (FIG. 6B) of antidote protein for clone #94 characterized in Example 3. Also depicted are Western blots using the anti-HC (heavy chain, FIG. 6C) antibody and anti-LC (light chain, FIG. 6D) antibody. BR (bench-scale reactor) 6 Feed 3 was added at day 2 and at day 4. Seeding density was 10×10⁵ cell/ml for BR6.

FIG. 7 shows SEQ ID NO: 1, the fXa derivative (also referred to as precursor r-Antidote) with the linker at amino acids 106-111.

FIG. 8 shows SEQ ID NO: 3, the fXa derivative (also referred to as r-Antidote) with the linker removed.

### DETAILED DESCRIPTION

### I. Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this disclosure.

The term “protein,” “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. In an alternative embodiment, the term “biological equivalent of” a polynucleotide refers to one that hybridizes under stringent conditions to the reference polynucleotide or its complement. Unless specifically recited herein, it is contemplated that any polynucleotide, polypeptide or protein mentioned herein also includes equivalents thereof. For example, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

“Hybridization” refers to hybridization reactions that can be performed under conditions of different “stringency.” Conditions that increase the stringency of a hybridization reaction are widely known and published in the art: see, for example, Sambrook, et al., infra. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours and washes of increasing duration, increasing frequency, or decreasing buffer concentrations.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology,” “identity,” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

“Polyfection” refers to a transfection technique based on a polymer, such as polyethylenimine (PEI).

When transfecting cells with DNA constructs, a non-coding carrier DNA may be transfected in addition to the DNA constructs carrying the genes of interest (i.e. Furin, selectable marker, and r-Antidote precursor). “Total transfected DNA” refers to the total amount of DNA (usually in μg) and includes plasmid DNA (or other DNA construct) and carrier DNA.

The term “fraction” when used in the context of protein isolation, refers to a collection of material separated based on a specific property. The specific property may include, by way of non-limiting example, size, mass, isolectric point, charge, and the like.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

The term “constructs” as used herein refers to artificial DNA fragments. These include, for example, plasmids, primers, cosmids, expression vectors, and the like.

The term “non-endogenous” refers to a polypeptide or polynucleotide non-native to the cell. A “non-endogenous” polynucleotide or polypeptide is typically one that has been introduced into the cell by gene transfer or protein administration. When the term “non-endogenous” applies to a polynucleotide, the polynucleotide may be located extrachromasomally or intrachromasomally (as an integrated piece of DNA into the host cells genome).

The term “endogenous” refers to a polypeptide or polynucleotide native to the cell (i.e., one that is naturally expressed or present in the cell).

The term “expression level” refers to the amount of protein present in the cell. The expression level may be defined in relation to another protein (either endogenously or non-endogenously expressed). Methods of determining the expression level of proteins are known in the art and are described herein.

“Factor Xa” or “fXa” or “fXa protein” refers to a serine protease in the blood coagulation pathway, which is produced from the inactive factor X (fX). Factor Xa is activated by either factor IXa with its cofactor, factor VIIIa, in a complex known as intrinsic Xase, or factor VIIa with its cofactor, tissue factor, in a complex known as extrinsic Xase. fXa forms a membrane-bound prothrombinase complex with factor Va and is the active component in the prothrombinase complex that catalyzes the conversion of prothrombin to thrombin. Thrombin is the enzyme that catalyzes the conversion of fibrinogen to fibrin, which ultimately leads to blood clot formation.

“des-Gla fXa” refers to fXa that does not have a Gla-domain. These fXa derivatives are described in U.S. Pat. No. 8,153,590, which is herein incorporated by reference in its entirety.

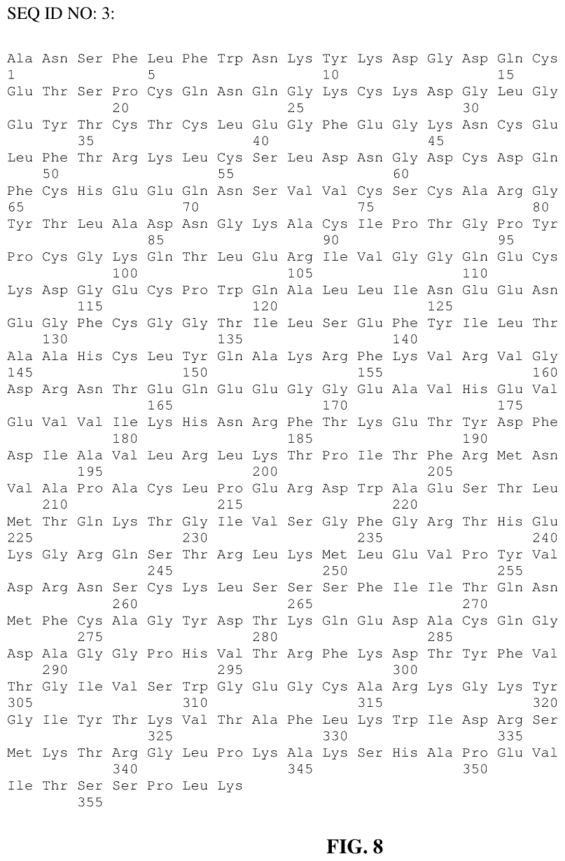

As used herein, “fXa derivatives” refer to modified fXa proteins that do not compete with fXa in assembling into the prothrombinase complex and have reduced or no procoagulant activities, and yet bind and/or substantially neutralize the anticoagulants, such as fXa inhibitors. Examples of fXa derivatives are provided in WO2009/042962, and further provided herein, such as SEQ ID NO: 3 (FIG. 8) and biological equivalents thereof.

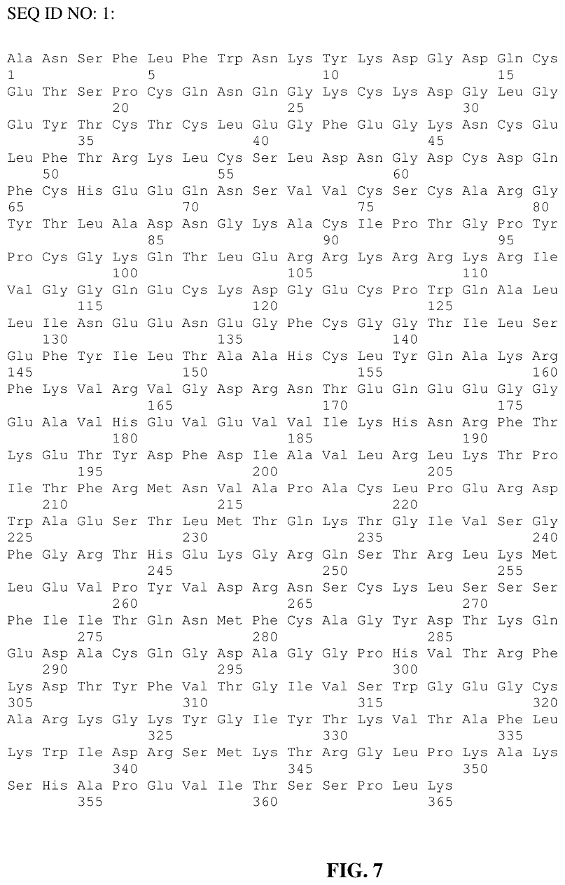

The term “Furin” or “paired basic amino acid cleaving enzyme,” as used herein, refers to a protein having an amino acid sequence substantially identical to any of the representative Furin sequences of GenBank Accession Nos. NP_002560 (human), NP_001074923 (mouse) or NP_062204 (rat). Suitable cDNA encoding Furin are provided at GenBank Accession Nos. NM_002569 (human), NM_001081454 (mouse) or NM_019331 (rat). In a particular aspect, Furin refers to a human Furin. A representative human Furin protein sequence is provided in SEQ ID NO: 2 (FIG. 7), and a representative human Furin cDNA sequence is provided in SEQ ID NO: 4 (FIG. 7).

“r-Antidote precursor” refers to the fXa derivative represented by SEQ ID NO: 1 which contains 3 mutations relative to fXa. The first mutation is the deletion of 6-39 aa in the Gla-domain of FX. The second mutation is replacing the activation peptide sequence 143-194 aa with -RKR-. This produces a -RKRRKR- (SEQ ID NO: 5) linker connecting the light chain and the heavy chain. Upon secretion, this linker is cleaved in CHO resulting in a cleaved two-chain polypeptide. Accordingly, the term “cleaved two-chain polypeptide” refers to a polypeptide of SEQ ID NO: 3, or a polypeptide having 80% identity to SEQ ID NO: 3, having two-chains and being linked together by at least one disulfide bond. The N-terminal chain consists of amino acids 1-105 of SEQ ID NO: 3 and the C-terminal chain consists of amino acids 106-359 of SEQ ID NO: 3. Optionally, the LC chain may contain 1, 2, 3, 4, 5 or 6 amino acid residues of the linker. Such additional residues result from the incomplete removal of the linker polypeptide. The third mutation is the mutation of active site residue 5379 to an Ala residue. This amino acid substitution corresponds to amino acid 296 and 290 of SEQ ID NOS: 1 and 3, respectively. The term “r-Antidote” refers to the polypeptide after cleavage and processing of the linker. This is represented by SEQ ID NO: 3.

The term “CHO” refers to Chinese hamster ovary cells.

“COS” refers to a cell line was obtained by immortalizing a CV-1 cell line derived from kidney cells of the African green monkey with a version of the SV40 genome that can produce large T antigen but has a defect in genomic replication. The word COS is an acronym, derived from the cells being CV-1 (simian) in Origin, and carrying the SV40 genetic material.

“BHK” refers to baby hamster kidney cells.

“HEK 293” refers to Human Embryonic Kidney 293 cells that were originally derived from human embryonic kidney cells grown in tissue culture.

The term “selectable marker” refers to a gene introduced into a cell that confers a trait suitable for artificial selection. They are a type of reporter gene used in laboratory microbiology, molecular biology, and genetic engineering to indicate the success of a transfection or other procedure meant to introduce foreign DNA into a cell. Selectable markers can include, by way of non-limiting example, antibiotic resistance genes such as, for example, genes that provide antibiotic resistance to puromycin, neomycin and hygromycin, and the like. The puromycin N-acetyl-transferase (PAC) gene confers resistance to puromycin. The neo gene provides resistance to neomycin, kanamycin, and geneticin. The hygromycin phosphotransferase gene (hph) provides resistance to hygromycin. Also included are genes such as dihydrofolate reductase (DHFR), or mutants thereof that provide resistance to methotrexate. The term “selectable marker” is also intended to describe a marker which allows researchers to distinguish between wanted and unwanted cells. Examples include genes that produce a protein with a distinguishing phenotype such as a pigment or fluorescence.

The term “antibiotic resistance” refers to a cell having the ability to survive exposure to an antibiotic. The concentration of the antibiotic is one that is known to eliminate cells that lack the antibiotic resistance gene and allows for cells with the antibiotic resistance gene to survive. Typically, cells with antibiotic resistance will maintain antibiotic resistance without continued selection. However, spontaneous mutations may result in a loss of resistance, in which case, additional selection or exposure to the antibiotic may be required to eliminate cells that have lost resistance.

The term “DNA construct” refers to DNA that contains a polynucleotide of interest and optionally other functional elements. Other functional elements may include, for example, an origin of replication, a selectable marker, a promoter, and a termination sequence.

“Extrachromosomal DNA” refers to DNA located or maintained in a cell apart from the chromosomes.

The term “integrated” when used in the context of a DNA construct refers insertion of the DNA construct into the host cell (i.e. isolated cell) genome.

A “plasmid” or “DNA plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

The term “cell culture media” refers to media used in the culturing of cells. The culture medium is designed to support the grown of the cell, and differs depending on the cell-type. It is within the knowledge of the skilled artisan to select the appropriate media based on the host cell type. Examples of typical cell culture techniques and media are described herein.

“Gene delivery,” “gene transfer,” “transducing,” “transfecting” and the like as used herein, are terms referring to the introduction of an exogenous polynucleotide (sometimes referred to as a “transgene”) into a host cell, irrespective of the method used for the introduction.

### II. Cells and Constructs

In vivo treatment of a human Factor Xa derivative (i.e., r-Antidote precursor) with Furin allows for the improved processing of a functional, 2-chain protein. Accordingly, aspects of the present disclosure relate to cells and constructs for the expression and processing of fXa derivatives.

The current disclosure provides cells and methods for the improved or enhanced processing of the one-chain r-Antidote precursor to the cleaved two-chain r-Antidote protein that acts as an antidote to fXa inhibitors. Accordingly, one embodiment of the present disclosure provides an isolated cell containing a first polynucleotide encoding a fXa derivative and a second polynucleotide encoding a Furin protein. The first and second polynucleotides, in one aspect, are on separate polynucleotide constructs. In another aspect, the first and second polynucleotides are on the same polynucleotide construct. Thus, another embodiment of the present disclosure provides a polynucleotide construct comprising the first and the second polynucleotides.

In one aspect, the fXa derivative has an amino acid sequence of SEQ ID NO: 1 or a polypeptide having at least 80% sequence identity to SEQ ID NO: 1. The fXa derivative represented by SEQ ID NO: 1 contains three mutations relative to fXa. The first mutation is the deletion of 6-39 aa in the Gla-domain of FX. The second mutation replaces the activation peptide sequence 143-194 aa with -RKR-. This produced a -RKRRKR- (SEQ ID NO: 5) linker connecting the light chain and the heavy chain. Upon secretion, this linker is cleaved in CHO resulting in a two-chain fXa molecule. The third mutation is mutation of active site residue 5379 to an Ala residue. This amino acid substitution corresponds to amino acid 296 and 290 of SEQ ID NOS: 1 and 3, respectively. The fXa derivative does not compete with fXa in assembling into the prothrombinase complex, but instead bind and/or substantially neutralize the anticoagulants, such as fXa inhibitors. The derivatives useful as antidotes are modified to reduce or remove intrinsic procoagulant and anticoagulant activities, while retaining the ability to bind to the inhibitors. Structurally, the derivatives are modified to provide either no procoagulant activity or reduced procoagulant activity. “Procoagulant activity” is referred to herein as an agent's ability to cause blood coagulation or clot formation. Reduced procoagulant activity means that the procoagulant activity has been reduced by at least about 50%, or more than about 90%, or more than about 95% as compared to wild-type fXa.

In another embodiment, the amino acid sequence having at least 80% sequence identity to SEQ ID NO: 3 has reduced procoagulant activity compared to wild-type factor Xa. In a further embodiment, the amino acid sequence having at least 80% sequence identity to SEQ ID NO: 3 does not assemble into a prothrombinase complex. In further embodiments, the amino acid sequence having at least 85%, at least 90%, at least 95%, or at least 98% sequence identity to SEQ ID NO: 3 has reduced procoagulant activity compared to wild-type factor Xa. In further embodiments, the amino acid sequence having at least 85%, at least 90%, at least 95%, or at least 98% sequence identity to SEQ ID NO: 3 does not assemble into a prothrombinase complex.

In one embodiment, the isolated cell further comprises a two-chain polypeptide comprising the amino acid sequence of SEQ ID NO: 3 or an amino acid sequence having at least 80% sequence identity to SEQ ID NO: 3.

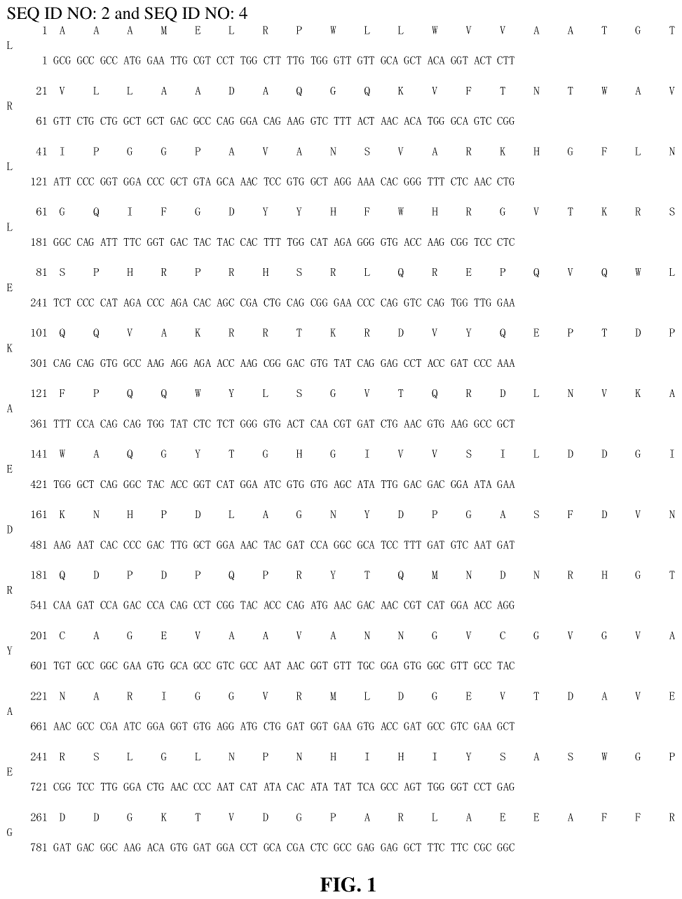

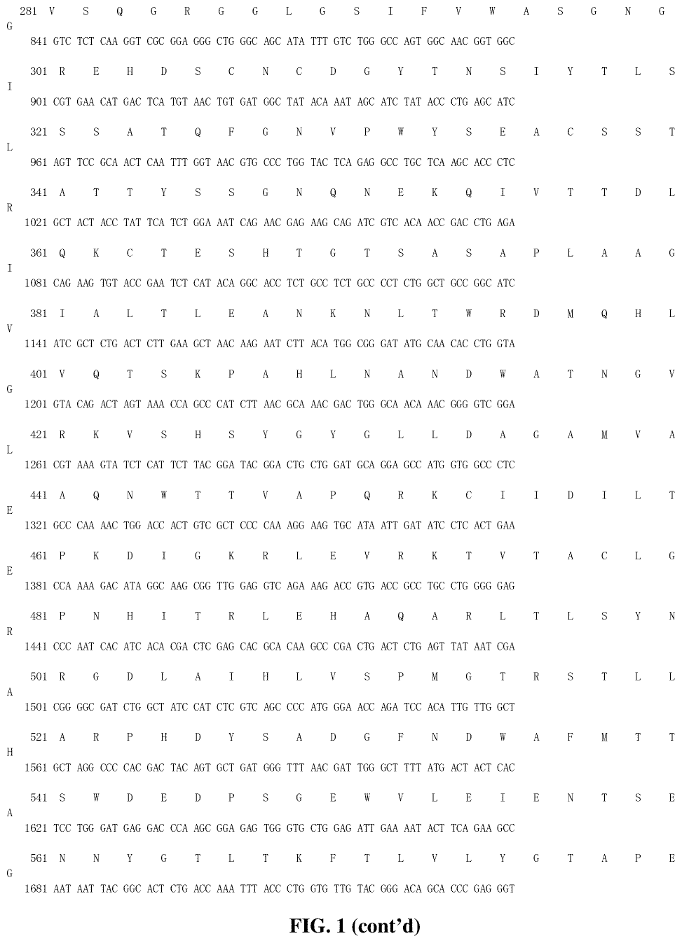

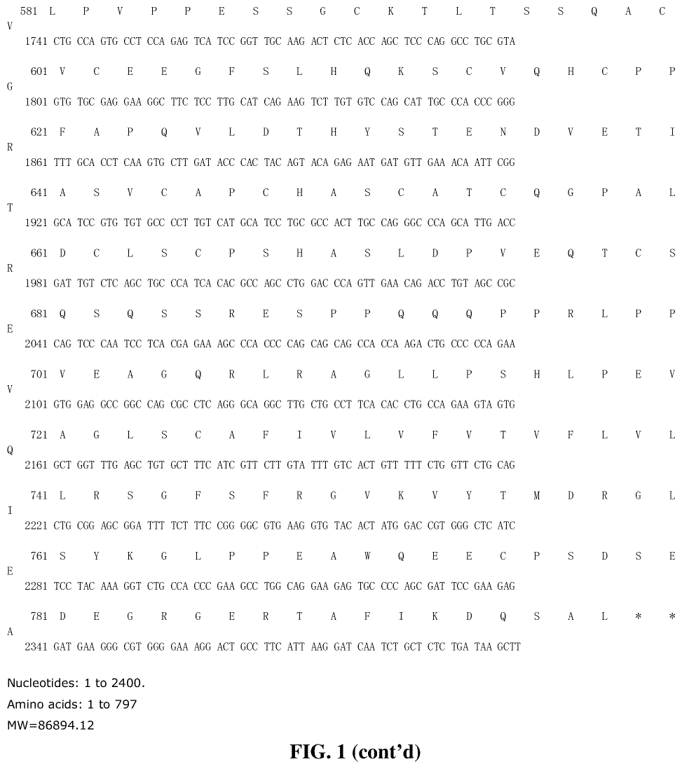

In one aspect, the Furin protein has an amino acid sequence of SEQ ID NO: 2 (FIG. 1).

In certain embodiments, the isolated cell described herein further comprises a selectable marker that may be expressed in the cell. In further embodiments, the selectable marker provides resistance to a compound selected from the group consisting of puromycin, methotrexate, neomycin and hygromycin. In one embodiment, the selectable marker provides resistance to methotrexate. In a further embodiment, the selectable marker provides resistance to puromycin. In another embodiment, the selectable marker provides antibiotic resistance to the cell.

The polynucleotides described herein can be contained on and/or expressed from a DNA construct. Examples of DNA constructs include plasmids, cosmids, expression vectors, phagemids, fosmids, and artificial chromosomes such as bacterial artificial chromosomes, yeast artificial chromosomes, and human artificial chromosomes. In certain embodiments, the first or second polynucleotides are on an extrachromosomal DNA construct. In another embodiment, the first or second polynucleotide is on a DNA construct integrated into the chromosomal DNA of the isolated cell. Stable cell lines with the expression vector integrated into its genome can allow for more stable protein expression in the cell population, resulting in more consistent results. The first and second polynucleotides may be contained on one DNA construct or separate constructs. When they are contained on one construct, they may use separate promoters for expression or the same promoter. Methods for expressing two proteins from one promoter are known in the art and include, for example, the use of an internal ribosome entry sequence (IRES).

The DNA constructs or plasmids containing the first and second polynucleotide can be transfected into the cell by a variety of methods known to those skilled in the art. In one embodiment, the DNA plasmids or constructs are transfected into the isolated cell by polyfection. In a further embodiment, the plasmid or DNA construct comprising the second polynucleotide is from about 1% to about 50% of total transfected DNA. Alternatively, the plasmid or DNA construct comprising the second polynucleotide is from about 1% to about 90% of total transfected DNA, or from about 1% to about 80%, or from about 1% to about 70%, or from about 1% to about 60%, or from about 1% to about 50%, or from about 1% to about 40%, or from about 1% to about 30%, or from about 1% to about 10%, or from about 3% to about 10% of total transfected DNA. In further embodiments, the plasmid or DNA construct comprising the second polynucleotide is about 3% of total transfected DNA, or about 5%, or about 10%, or about 15%, or about 20%, or about 25%, or about 30%, or about 35%, or about 40%, or about 45%, or about 50%, or about 60% of total transfected DNA.

Cells of the present disclosure can be prepared by introducing the first polynucleotide and the second polynucleotide into a cell or tissue using a gene delivery vehicle. Methods for gene delivery include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

In certain embodiments, the polynucleotides are introduced to a cell by transfection. Transfection techniques are well known in the art and can include chemical-based transfection, such as calcium phosphate transfection and polyfection, and non chemical-based transfection such as electroporation, optical transfection, and gene electrotransfer. Also included are lipofection techniques. Lipofection generally uses a positively charged (cationic) lipid to form an aggregate with the negatively charged (anionic) genetic material. A net positive charge on this aggregate has been assumed to increase the effectiveness of transfection through the negatively charged phospholipid bilayer.

In one aspect, introduction of the first polynucleotide is performed before the introduction of the second polynucleotide. In another aspect, introduction of the first polynucleotide is performed after the introduction of the second polynucleotide. In yet another aspect, both the first and the second polynucleotides are co-incubated with a cell. In a particular aspect, the first and second polynucleotides are on the same construct and thus the introduction is carried out simultaneously.

In further embodiments, the isolated cell described herein further comprises a first polypeptide comprising the amino acid sequence of SEQ ID NO: 1 or a polypeptide having at least about 80% sequence identity to SEQ ID NO: 1 and a second polypeptide comprising the amino acid sequence of SEQ ID NO: 3 or a polypeptide having at least about 80% sequence identity to SEQ ID NO: 3. Inefficient cleavage of the peptide results in single chain polypeptide of SEQ ID NO: 1 or a polypeptide having at least about 80% sequence identity to SEQ ID NO: 1. The methods and isolated cells described herein provide for improved efficiency in cleavage of the fXa derivative. Therefore, the ratio of the two-chain polypeptides having SEQ ID NO: 3, 80% homology to SEQ ID NO: 3 or SEQ ID NO: 3 containing linker residues to polypeptides of SEQ ID NO: 1 or a polypeptide having at least about 80% sequence identity to SEQ ID NO: 1 may be at least about 9:1 in certain embodiments. Alternatively, the ratio may be at least about 7:3, 8:2, 95:5, or 99:1.

In cells described herein, Furin is produced at an expression level higher than the endogenous expression level of the cell. Embodiments of the disclosure relate to isolated cells as described herein further comprising a polypeptide comprising the amino acid sequence of SEQ ID NO: 2 or a polypeptide having at least about 80% sequence identity to SEQ ID NO: 2. In a related embodiment, the expression level of polypeptide comprising the amino acid sequence of SEQ ID NO: 2 is at least 3 times the expression level of endogenous Furin. In further embodiments, the expression level of polypeptide comprising the amino acid sequence of SEQ ID NO: 2 is at least 4 times, at least 5 times, at least 6 times, at least 7 times, at least 8 times, at least 9 times, or at least 10 times the expression level of endogenous Furin.

The proteins can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In certain embodiments, the cell is a yeast cell or mammalian cell. The cells can be purchased from a commercial vendor such as the American Type Culture Collection (ATCC, Rockville Md., USA) or cultured from an isolate using methods known in the art. Examples of suitable eukaryotic cells include, but are not limited to HEK 293 cells, the hamster cell line BHK-21, CHO cells; murine cell lines such as NIH3T3, NS0, and C127; simian cell lines such as COS and Vero; and human cell lines such as HeLa, PER.C6 (commercially available from Crucell), U-937, and Hep G2. In certain embodiments, the mammalian cell is a cell-type selected from the group consisting of CHO, COS, BHK, and HEK 293. In a further embodiment, the cell-type is CHO. In yet a further embodiment, the cell is a CHO cell subtype selected from the group consisting of K, M and DG44. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### III. Methods of Preparing fXa Derivatives

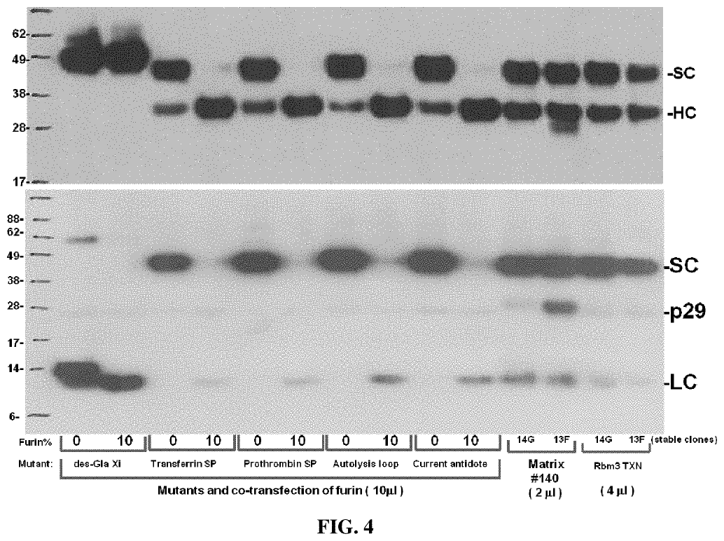

Previous methods for preparing functional r-Antidote from cell lines expressing the r-Antidote precursor protein led to reduced yields of functional r-Antidote due to inefficient cleavage of the precursor protein. The in vivo co-expression of both the r-Antidote precursor (SEQ ID NO: 1) and Furin allows for efficient cleavage of the precursor to the functional two-chain r-Antidote protein (SEQ ID NO: 3). Furthermore, the in vivo co-expression of both r-Antidote precursor protein and Furin allows for the increased expression as well as increased function of the r-Antidote protein from the cell. In certain embodiments, about 70% of the r-Antidote precursor is cleaved. In other embodiments, about 75%, 80%, or 85% of the r-Antidote precursor is cleaved. In a preferred embodiment, about 90% or more is cleaved. In a more preferred embodiment, about 95% or more is cleaved. In yet another preferred embodiment, about 99% or more is cleaved. The amount of Furin expressed in the cell is an amount that allows for at least about 70% cleavage of the single-chain polypeptide to the two-chain polypeptide. Alternatively, the expression level of Furin is one that allows for at least about 75%, 80%, 85%, 90%, 95% or 99% cleavage of the single-chain polypeptide. The cleavage is not only dependent on the linker but also on the sequences surrounding the linker. Example 3 (FIG. 4) demonstrates that not every fX derivative improves the efficiency of linker cleavage.

Methods of preparing processed fXa derivatives are also provided. In one aspect, the methods entail expressing the fXa derivative and the Furin protein in the cell of the present disclosure. In another aspect, the methods further allow the expressed fXa derivative to be cleaved by the Furin protein in the cell.

By virtue of the cleavage, an unprocessed single chain fXa protein becomes a two chain polypeptide. This protein is cleaved by Furin, which is also known as PACE (Paired basic Amino acid Cleaving Enzyme). Furin cleaves proteins just downstream of a basic amino acid target sequence (canonically, Arg-X-(Arg/Lys)-Arg; SEQ ID NO: 6).

A polypeptide of SEQ ID NO: 3 or a polypeptide having at least 80% sequence identity to SEQ ID NO: 3 refers to the cleaved two-chain fXa derivative protein that acts as an antidote to inhibitors of fXa. This protein is processed and cleaved, which results in the removal of the -RKRRKR- (SEQ ID NO: 5) linker sequence. The linker sequence corresponds to amino acid numbers 106-111 of SEQ ID NO: 1. In certain embodiments, cleavage may occur without the complete removal of the linker sequences. Therefore, the cleaved two chain polypeptide may comprise SEQ ID NO: 3 with 1, 2, 3, 4, 5 or 6 linker amino acids after amino acid 105 of SEQ ID NO: 3. Upon cleavage, the two chain fXa derivative remains connected due to the disulfide bond between the two chains.

In another embodiment, the amino acid sequence having at least 80% sequence identity to SEQ ID NO: 3 has reduced procoagulant activity compared to wild-type factor Xa. In a further embodiment, the amino acid sequence having at least 80% sequence identity to SEQ ID NO: 3 does not assemble into a prothrombinase complex. In further embodiments, the amino acid sequence having at least 85%, at least 90%, at least 95%, or at least 98% sequence identity to SEQ ID NO: 3 has reduced procoagulant activity compared to wild-type factor Xa. In further embodiments, the amino acid sequence having at least 85%, at least 90%, at least 95%, or at least 98% sequence identity to SEQ ID NO: 3 does not assemble into a prothrombinase complex.

Further embodiments of the method aspects disclosed herein further comprise isolating, from the cell, a protein fraction comprising a polypeptide having at least about 80% sequence identity to SEQ ID NO: 3. In a related embodiment, the isolated protein fraction further comprises a polypeptide having at least about 80% sequence identity to SEQ ID NO: 1. Polypeptides having SEQ ID NO: 3, 80% homology to SEQ ID NO: 3 or SEQ ID NO: 3 containing linker residues represent the cleaved two-chain polypeptide, which is the functional protein. Inefficient cleavage of the peptide results in single chain polypeptide of SEQ ID NO: 1 or a polypeptide having at least about 80% sequence identity to SEQ ID NO: 1. The methods and isolated cells described herein provide for improved efficiency in cleavage of the fXa derivative. Therefore, the ratio of the two-chain polypeptides having SEQ ID NO: 3, 80% homology to SEQ ID NO: 3 or SEQ ID NO: 3 containing linker residues to polypeptides of SEQ ID NO: 1 or a polypeptide having at least about 80% sequence identity to SEQ ID NO: 1 may be at least about 9:1 in certain embodiments. Alternatively, the ratio may be at least about 7:3, 8:2, 95:5, or 99:1.

In cells described herein, Furin is produced at an expression level higher than the endogenous expression level of the cell. Embodiments of the disclosure relate to isolated cells as described herein further comprising a polypeptide comprising the amino acid sequence of SEQ ID NO: 2 or a polypeptide having at least about 80% sequence identity to SEQ ID NO: 2. In a related embodiment, the expression level of polypeptide comprising the amino acid sequence of SEQ ID NO: 2 is at least 3 times the expression level of endogenous Furin. In further embodiments, the expression level of polypeptide comprising the amino acid sequence of SEQ ID NO: 2 is at least 4 times, at least 5 times, at least 6 times, at least 7 times, at least 8 times, at least 9 times, or at least 10 times the expression level of endogenous Furin.

In another embodiment, the present disclosure provides a preparation of two chain fXa derivative prepared with cells, constructs or methods described herein.

The cleaved fXa derivative may be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, mixed-mode resins, exclusion chromatography, polyacrylamide gel electrophoresis, affinity chromatography, or isoelectric focusing. A particularly efficient method of purifying peptides is fast protein liquid chromatography or even HPLC.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG (polyethylene glycol), antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Example 1

### Development of Parental Stable Cell Lines Expressing the r-Antidote Protein

The r-Antidote producing cell line is a Chinese Hamster Ovary (CHO) clone which was stably transfected first with an expression vector containing the r-Antidote cDNA, resulting in a parental clone. The parental clone was further transfected with a full length human Furin cDNA in a separate vector (“Furin super-transfection”) to improve processing of the -RKRRKR- (SEQ ID NO: 5) linker in the r-Antidote precursor. The amino acid sequence of r-Antidote and the DNA sequence of expression vector have been described.

The host cell line used to produce the r-Antidote protein was the dihydrofolate reductase (DHFR)-deficient CHO-DUX B11 cell line. It was transfected with the expression vector encoding r-Antidote using a cationic liposome transfection agent (Lipofectamine 2000). “Subpools” of the transfection pool were cultured with stepwise increases of methotrexate (0, 50, 250, and 500 nM); subpools adapted to 500 nM methotrexate were adapted to suspension culture in a commercial serum free medium (CDM4CHO, available from Hyclone, Logan, Utah). The subpools which exhibited the best growth and product expression were sub-cloned. Clones were screened for growth and productivity. Research cell banks (RCBs) were made in CDM4CHO medium from the best three sub-clones (13F5-3C11, 14G1-3A4, 14G1-6A8) and tested for sterility and the absence of Mycoplasma.

13F5-3C11 and 14G1-6A8 cell lines were selected for initial cell culture development and Furin super-transfection. Clone 14G1-6A8 that was stably transfected with a full length human Furin cDNA was eventually selected as the final cell line for r-Antidote production.

### Example 2

### Transient Transfection with Furin-Containing Vector

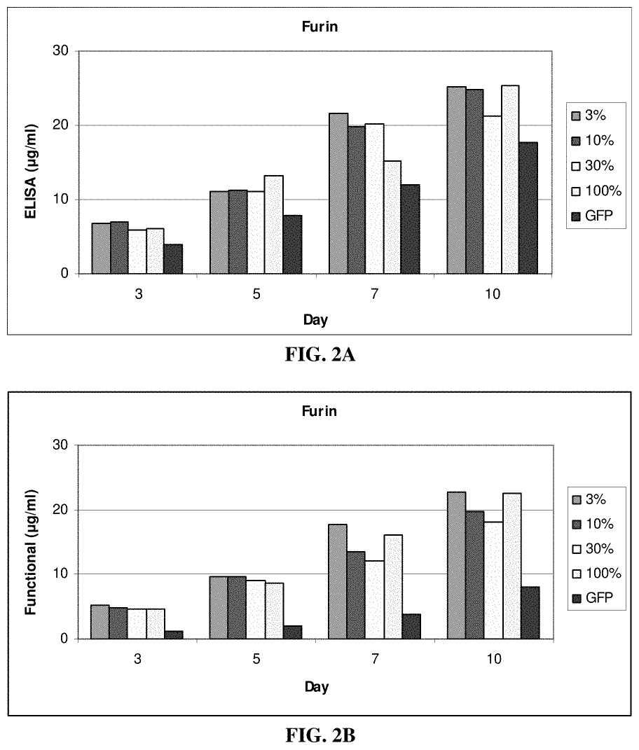

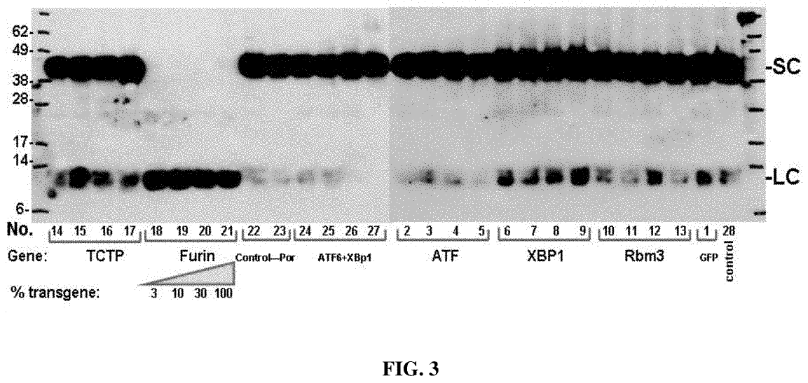

In order to evaluate the effect of cellular factors on r-Antidote expression and processing, 14G1 clone (14G1-6A8) in ProCHO medium was transiently transfected with a vector containing either Furin, Rbm3 (Putative RNA-binding protein 3), XBP1 (X-box binding protein 1), ATF6 (Activating transcription factor 6), or TCTP (translationally controlled tumour protein) cDNA (complimentary deoxyribonucleic acid). In some cases, two of these vectors were co-transfected to test their combined effect. r-Antidote expression level and quality were examined following the transient transfection on day 3, 5, 7 and 10. Interestingly, only Furin transfection improved the total percentage of functional protein, possibly due to enhanced processing of the -RKRRKR- (SEQ ID NO: 5) linker, or relief of an intracellular bottleneck for processing and secretion. FIG. 1 shows an optimized full length human Furin cDNA and translated amino acid sequence. FIG. 2 shows the protein expression level and functional activity. FIG. 3 shows the protein quality as assessed by Western Blots indicating that transfection of Furin completely eliminated the single-chain r-Antidote precursor, while other examples tested had no effect on the amount of single-chain r-Antidote precursor that was present.

Surprisingly, co-transfection of Furin with an alternative fX derivative des-Gla Xi, which was disclosed previously (US Patent Application No 2010-0255000), did not improve the -RKRRKR- (SEQ ID NO: 5) linker cleavage as shown in FIG. 4. These results indicate that cleavage of the -RKRRKR- (SEQ ID NO: 5) linker by Furin is dependent upon two factors, -RKRRKR- (SEQ ID NO: 5) linker and the amino acid sequence flanking the linker. Furthermore, it has previously been shown that replacing the -RKRRKR- (SEQ ID NO: 5) linker with -RKR- in the same fXa derivative construct did not produce properly processed two-chain molecule (see, for e.g., U.S. Pat. No. 5,968,897). Based on these preliminary findings, the 14G1 clone was further subjected to transfection with the Furin-vector under puromycin selection for generation of a stable cell line.

### Example 3

### r-Antidote Producing Cell Line with Stably Transfected Human Furin cDNA

The r-Antidote production cell line was generated by transfecting the 14G1-6A8 cell line with a vector containing a full length human Furin cDNA. A second vector containing the puromycin selection marker was co-transfected for clone selection.

Briefly, the parental stable cell line (14G1-6A8), which contained the r-Antidote expression vector and was generated in CDM4CHO medium, was first adapted to ProCHO5 medium (available commercially from Lonza, Cat #BE12-766Q) during the initial cell culture development process. Clone 14G1-6A8 was maintained in ProCHO5 medium with MTX (Methotrexate, 500 nM) prior to transfection of the vector containing an optimized full length human Furin cDNA.

The Furin-containing vector was co-transfected with a puromycin selection vector. Co-transfection was carried out in ProCHO5 medium without MTX by a chemical method based on a polymer (polyfection). An optimal ratio (w/w) of plasmid DNA used in the chemical transfection was 10% Furin-vector plasmid:10% purimycin-vector plasmid:80% carrier DNA.

The co-transfected cells were maintained in puromycin (15 μg/mL) for 10 days. At the end of the selection process, pools of transfected cells with good growth performance in the presence of the selective agent were obtained. Cells from each pool were frozen as back up.

Single-cell cloning was performed by limiting dilution (1 cell/well) of pools into 96-wells plates in 100 μL ProCHO5 medium without puromycin. Individual clones were selected based on r-Antidote expression level, functional activity and Western blot.

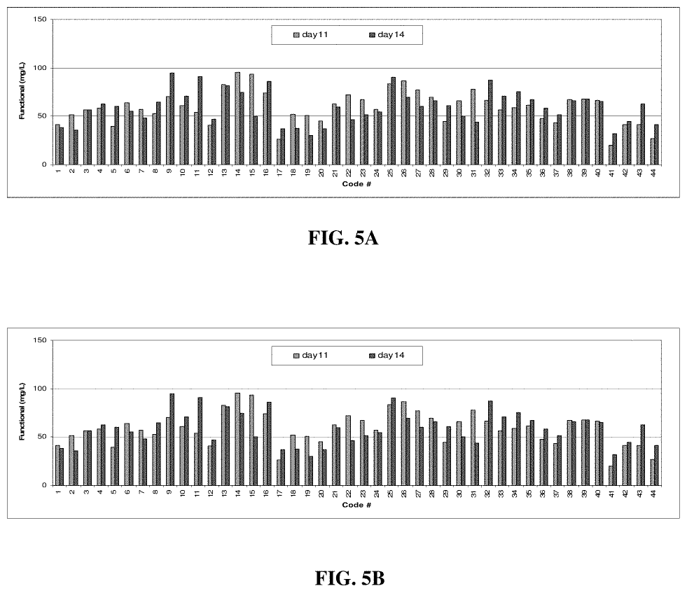

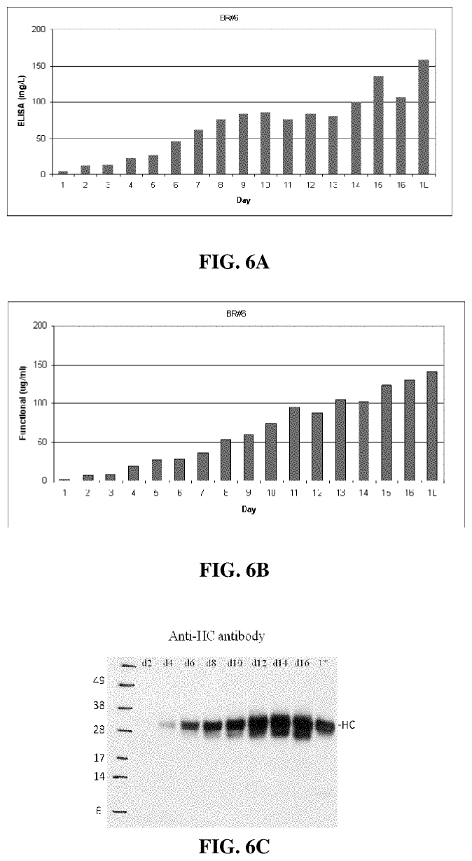

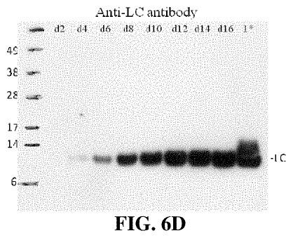

Subsequently, candidate clones were expanded and screened in a small spin-tube cultures and cultured for 6 days in ProCHO5. Based on protein expression level and quality, a sub-set of 10 clones was selected for a matrix study testing different culture conditions, from which four candidate clones (clone #92, #94, #126 and #127) were selected and RCBs were created. Growth of clones #92 and #94 were further tested in matrix experiment (FIG. 5) and 1.5 L bioreactors (FIG. 6 A, B). Clone #94 was eventually selected as the final clone for r-Antidote production.

The RCBs were created after a total of 10 passages for clone #92, #94, #126 and #127 in ProCHO5 medium without MTX or puromycin following the initial cell expansion from the candidate clones in 96-wells plates. 10% DMSO+90% ProCHO5 medium without MTX or puromycin was used as the freeze medium for the RCBs (1 mL/vial, 30×10⁶ cells/mL).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

Other embodiments are within the following claims. In addition, where features or aspects of the disclosure are described in terms of Markush groups, those skilled in the art will recognize that the disclosure is also thereby described in terms of any individual member or subgroup of members of the Markush group.

1. An isolated cell comprising:
a first polynucleotide encoding a polypeptide comprising the amino acid sequence of SEQ ID NO: 1 or a polypeptide having at least about 80% sequence identity to SEQ ID NO: 1 and
a second non-endogenous polynucleotide encoding a polypeptide comprising the amino acid sequence of SEQ ID NO: 2 or a polypeptide having at least about 80% sequence identity to SEQ ID NO: 2.

2-4. (canceled)

5. The isolated cell of claim 4, wherein the cell-type is CHO.

6. The isolated cell of claim 5, wherein the cell is a CHO cell subtype selected from the group consisting of K, M and DG44.

7-8. (canceled)

9. The isolated cell of claim 1, further comprising a selectable marker.

10. The isolated cell of claim 9, wherein the selectable marker provides resistance to a compound selected from the group consisting of puromycin, methotrexate, neomycin and hygromycin.

11. The isolated cell of claim 10, wherein the selectable marker provides resistance to methotrexate.

12. The isolated cell of claim 10, wherein the selectable marker provides resistance to puromycin.

13. The isolated cell of claim 12, wherein the selectable marker provides antibiotic resistance to the cell.

14. The isolated cell of claim 1, wherein the first or second polynucleotide is on an extrachromosomal DNA construct.

15. The isolated cell of claim 1, wherein the first or second polynucleotide is on a DNA construct integrated into the chromosomal DNA of the isolated cell.

16. The isolated cell of claim 1, wherein the first and second polynucleotides are on one DNA plasmid.

17. The isolated cell of claim 1, wherein the wherein the first and second polynucleotides are on different DNA plasmids.

18. The isolated cell of claim 1 further comprising
a first polypeptide comprising the amino acid sequence of SEQ ID NO: 1 or a polypeptide having at least about 80% sequence identity to SEQ ID NO: 1 and
a second polypeptide comprising the amino acid sequence of SEQ ID NO: 3 or a polypeptide having at least about 80% sequence identity to SEQ ID NO: 3.

19. The isolated cell of claim 18, wherein the ratio of the second polypeptide to the first polypeptide is at least about 8:2.

20. The isolated cell of claim 18, wherein the ratio of the second polypeptide to the first polypeptide is at least about 9:1.

21. The isolated cell of claim 1 further comprising a polypeptide comprising the amino acid sequence of SEQ ID NO: 2 or a polypeptide having at least about 80% sequence identity to SEQ ID NO: 2.

22. The isolated cell of claim 21, wherein expression level of the polypeptide comprising the amino acid sequence of SEQ ID NO: 2 is at least 3 times expression level of endogenous Furin.

23. A composition comprising the isolated cell of claim 1 and cell culture media.

24. A method of preparing a cleaved two chain polypeptide comprising the amino acid sequence of SEQ ID NO: 3 or a polypeptide having at least about 80% sequence identity to SEQ ID NO: 3, wherein the method comprises expressing in an isolated cell:
a first polynucleotide encoding a polypeptide comprising the amino acid sequence of SEQ ID NO: 1 or a polypeptide having at least about 80% sequence identity to SEQ ID NO: 1 and
a second non-endogenous polynucleotide encoding a polypeptide comprising the amino acid sequence of SEQ ID NO: 2 or a polypeptide having at least about 80% sequence identity to SEQ ID NO: 2.

25-52. (canceled)

53. A polynucleotide construct that comprises
a first polynucleotide encoding a polypeptide comprising the amino acid sequence of SEQ ID NO: 1 or a polypeptide having at least about 80% sequence identity to SEQ ID NO: 1; and
a second polynucleotide encoding a polypeptide comprising the amino acid sequence of SEQ ID NO: 2 or a polypeptide having at least about 80% sequence identity to SEQ ID NO: 2.
